(19) United States
(12) Patent Application Publication
(10) Pub. No.: US 2013/0220835 A1
(43) Pub. Date: Aug. 29, 2013

(54) POLYMERIC VDAT NANOPARTICLES FOR USE IN BIOSENSORS

(71) Applicants: Zuifang LIU, Inverness (GB); Louise SIMPSON, Inverness (GB); Marco CARDOSI, Inverness (GB)

(72) Inventors: Zuifang LIU, Inverness (GB); Louise SIMPSON, Inverness (GB); Marco CARDOSI, Inverness (GB)

(73) Assignee: LifeScan Scotland Limited

(21) Appl. No.: 13/408,970

(22) Filed: Feb. 29, 2012

(51) Int. Cl. G01N 27/327; G01N 33/66; G01N 33/50; B82Y 30/00

(52) U.S. Cl. 205/782; 204/403.01; 204/403.04; 205/775; 205/792; 977/773

(57) ABSTRACT

A biosensor (such as an electrochemical-based analytical test strip configured for the determination of glucose in a whole blood sample) includes a substrate, an electrode disposed on the substrate and a uric acid scavenger layer containing polymeric vinyl-4,6-diamino-1,3,5-triazine (polyVDAT) nanoparticles. Aqueous compositions useful in, for example, the manufacturing of such biosensors include polyVDAT nanoparticles and water with the polyVDAT nanoparticles being present as a dispersion in the water. A method for determining an analyte in a bodily fluid sample containing uric acid includes applying a bodily fluid sample containing uric acid to a biosensor such that the bodily fluid sample comes into contact with a uric acid scavenger layer containing polymeric vinyl-4,6-diamino-1,3,5-triazine (polyVDAT) nanoparticles and determining the analyte based on an electronic signal produced by the biosensor.

### BACKGROUND OF THE INVENTION

1. Field of the Invention

The present invention relates, in general, to medical devices and, in particular, to polymeric nanoparticle compositions, biosensors containing polymeric nanoparticles and related methods.

2. Description of Related Art

The determination (e.g., detection and/or concentration measurement) of an analyte in a fluid sample is of particular interest in the medical field. For example, it can be desirable to determine glucose, ketone bodies, cholesterol, lipoproteins, triglycerides, acetaminophen and/or HbA1c concentrations in a sample of a bodily fluid such as urine, blood, plasma or interstitial fluid. Such determinations can be achieved using sensors, based on, for example, visual, photometric or electrochemical techniques. Conventional electrochemical-based analytical test strips are described in, for example, U.S. Pat. Nos. 5,708,247 and 6,284,125, each of which is hereby incorporated in full by reference.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention, in which:

FIG. 1 is a simplified chemical sequence depicting a free-radical synthesis of polyVDAT (i.e., polymeric vinyl-4,6-diamino-1,3,5-triazine) nanoparticles as can be employed in embodiments of the present invention;

FIG. 2 is a simplified chemical structure depiction of a polyVDAT nanoparticle of FIG. 1 hydrogen-bonding with (scavenging) a uric acid molecule;

FIG. 3 is a Scanning Electron Microscopy (SEM) image of polyVDAT nanoparticles synthesized in Example 1 as described herein;

FIG. 4 is a SEM image of polyVDAT nanoparticles synthesized in Example 2 as described herein;

FIG. 5 depicts linear sweep voltammograms of uric acid in PBS before and after mixing with polyVDAT nanoparticles;

FIGS. 6A and 6B are graphs of an electrochemical response current versus uric acid concentration (FIG. 6A) and versus glucose concentration (FIG. 6B) for a biosensor that includes polyVDAT nanoparticles and a biosensor that includes polystyrene nanoparticles;

FIG. 7 is a simplified exploded perspective view of an analytical test strip containing a uric acid scavenger layer containing polyVDAT nanoparticles according to an embodiment of the present invention;

FIG. 8 is a sequence of simplified depictions of an analytical test strip that includes a uric acid scavenger layer containing polyVDAT nanoparticles disposed above an enzymatic reagent layer during use according to an embodiment of the present invention in use;

FIG. 9 is a sequence of simplified depictions of an analytical test strip that includes a combined uric acid scavenger layer containing polyVDAT nanoparticles and enzymatic reagent layer during use according to an embodiment of the present invention in use;

FIG. 10 is a sequence of simplified depictions of an analytical test strip that includes a uric acid scavenger layer containing polyVDAT nanoparticles disposed under an enzymatic reagent layer during use according to an embodiment of the present invention in use; and

FIG. 11 is a flow diagram depicting stages in a method for determining an analyte in a bodily fluid sample containing uric acid according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein

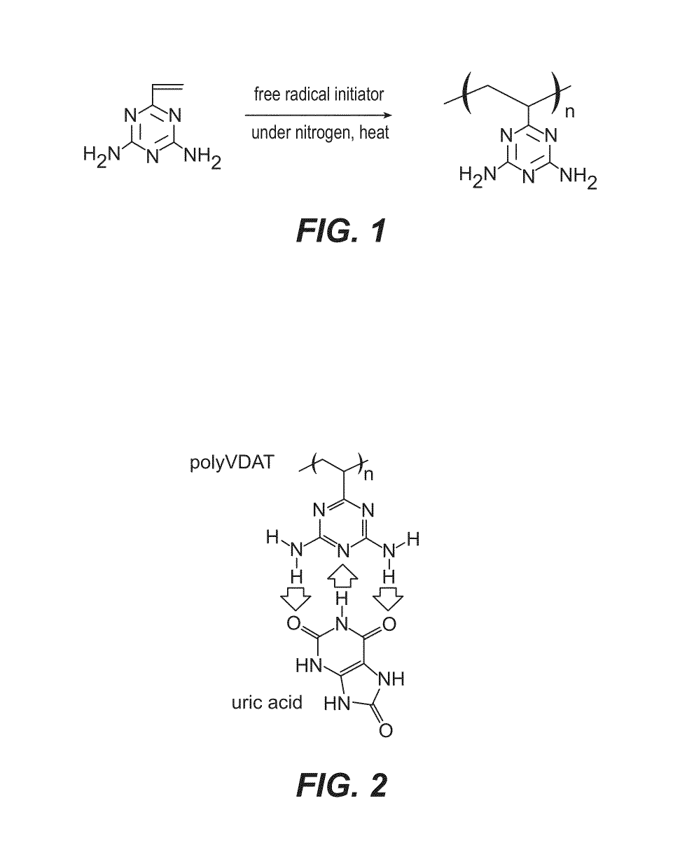

In general, biosensors (such as an electrochemical-based analytical test strip configured for the determination of glucose in a whole blood sample) according to embodiments of the present invention include a substrate, an electrode disposed on the substrate and a uric acid scavenger layer containing polymeric nanoparticles that include polymerized vinyl-4,6-diamino-1,3,5-triazine (also referred to herein as polyVDAT nanoparticles). The polyVDAT nanoparticles included in biosensors according to embodiments of the present invention can include only polymerized vinyl-4,6-diamino-1,3,5-triazine (i.e., vinyl-4,6-diamino-1,3,5-triazine molecules polymerized directly to other vinyl-4,6-diamino-1,3,5-triazine molecules as depicted in FIGS. 1 and 2 and described herein), vinyl-4,6-diamino-1,3,5-triazine copolymerized with other suitable monomers such as styrene and methyl methacrylate, and/or vinyl-4,6-diamino-1,3,5-triazine cross-linked by a suitable crosslinking compound(s) such as, for example, divinylbenzene. In this regard, cross-linked vinyl-4,6-diamino-1,3,5-triazine refers to a three dimensional covalently linked molecular polymeric network. It should be noted, however, that the use of polyVDAT nanoparticles that contain only polymerized vinyl-4,6-diamino-1,3,5-triazine (see, for examples FIGS. 1 and 2 and the associated descriptions herein) are preferred since the surface density of VDAT functionalities on such polyVDAT nanoparticles is maximized, hence maximizing their uric acid scavenging capabilities.

Biosensors according to embodiments of the present invention are beneficial in that the uric acid scavenging layer reduces the interfering effect of uric acid in a bodily fluid sample applied to the biosensor, thus increasing the accuracy of the biosensor. Uric acid can behave as an interferent by, for example, exhibiting either direct electroactive behavior at the electrode of the biosensor or by being oxidized by enzymatic reagents (such as ferricyanide) included in the biosensor. Such interfering effects are mitigated once uric acid is bound to the polyVDAT nanoparticles through hydrogen bonding (i.e., scavenged).

In general, aqueous vinyl-4,6-diamino-1,3,5-triazine compositions according to embodiments of the present invention include polyVDAT nanoparticles and water with the polyVDAT nanoparticles being present as a dispersion in the water. Typically, to avoid nanoparticle agglomeration during the nanoparticle synthesis, such aqueous vinyl-4,6-diamino-1,3,5-triazine compositions include polyVDAT nanoparticles at a w/w % of no more than 5%. However, the w/w % of polyVDAT can exceed 5% if deleterious agglomeration does not occur during/or after the nanoparticle synthesis. Aqueous vinyl-4,6-diamino-1,3,5-triazine compositions according to embodiments of the present invention are particularly advantageous in comparison to non-aqueous compositions due to their simplicity, the ability to readily add further components such as the commercially available binder Pluronic P103, and their compatibility with aqueous enzymatic reagents commonly used in biosensor manufacturing.

A method for determining an analyte in a bodily fluid sample containing uric acid according to embodiments of the present invention includes applying a bodily fluid sample (such as a whole blood sample) containing uric acid to a biosensor such that the bodily fluid sample comes into contact with a uric acid scavenger layer containing polymeric vinyl-4,6-diamino-1,3,5-triazine (polyVDAT) nanoparticles, and determining the analyte based on an electronic signal produced by the biosensor.

The term “nanoparticle” as used herein refers to particles that are of a size, or have a structural feature of a size, that causes them to display properties or behaviors that are different than the properties of the bulk material. For example, polyVDAT nanoparticles according to embodiments of the present invention can be formulated as a free-flowing dispersion in a liquid (e.g., water) without changing their dimensions or shape.

The term “dispersion” as used herein refers to a mixture, in which fine particles of one or more than one substance (for example, polyVDAT nanoparticles) are scattered throughout another substance or mixture of substances (for example, water). Dispersions are classed as suspensions.

The term “biosensor” as used herein refers to an analytical device that includes a biological material (e.g., an enzyme) associated or integrated with a physiochemical transducer system (such as an electrochemical-based system). Examples include immune-sensors, enzyme-based biosensors (such as electrochemical-based analytical test strips configured for the determination of an analyte in a whole blood sample) and whole-cell based biosensors. Such biosensors typically produce an electronic signal that is proportional to the concentration of a predetermined analyte or group of analytes.

Uric acid is a known interferent for electrochemical-based biosensors. Moreover, the concentration of uric acid in bodily fluid samples (e.g., blood samples and plasma samples) can vary from person to person based on their gender, health and medications. Therefore, the presence of uric acid in a bodily fluid sample applied to a biosensor can lead to inaccuracies in biosensor results. PolyVDAT can scavenge uric acid via hydrogen bonding in biological fluids at a neutral/or physiological pH. However, polyVDAT bulk material is only water soluble at low (acidic) pH (<4.0) and is, therefore, not compatible with typical biosensors or their manufacturing processes.

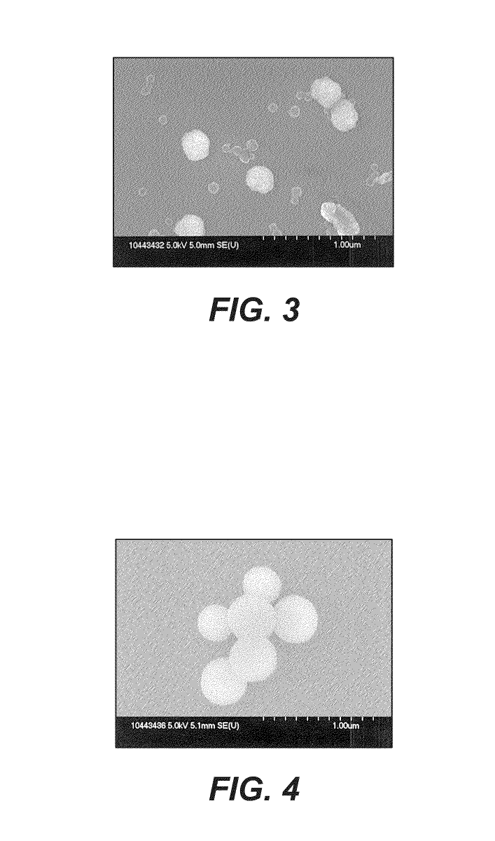

FIG. 1 is a simplified chemical sequence depicting a free-radical synthesis of polyVDAT (i.e., polymeric vinyl-4,6-diamino-1,3,5-triazine) nanoparticles. FIG. 2 is a simplified chemical structure depiction of polyVDAT hydrogen-bonding with a uric acid molecule. FIG. 3 is a Scanning Electron Microscopy (SEM) image of irregular-shaped polyVDAT nanoparticles synthesized in Example 1 as described herein. FIG. 4 is a SEM image of essentially spherical polyVDAT nanoparticles synthesized in Example 2 as described herein.

Referring to FIGS. 1-4, it has been determined that polyVDAT nanoparticles created via an emulsifier-free emulsion polymerization (such as that depicted in FIG. 1) can be employed in stable aqueous dispersions at biosensor relevant pHs (typically in the pH range of 4 to 14) and can scavenge uric acid via hydrogen bonding (see FIG. 2). Such nanoparticles have a diameter in the range of 30 nanometers to 1000 nanometers (see the example of FIGS. 3 and 4), and an “n” value in the range of, for example, 15 to 5000. Assuming a polyVDAT density of 1.35 g/cm³, a spherical polyVDAT nanoparticle according to the equation of FIG. 1 with a mono-distributed diameter of 1000 nanometers would have a minimum surface area of 4.44×10⁴ cm²/gram. The minimum “n” value can be predetermined to provide a polymer that precipitates out of solution to form polyVDAT nanoparticles

PolyVDAT nanoparticles created via the emulsifier-free emulsion polymerization have nanoparticle surfaces with the VDAT functional groups exposed (which is beneficial for hydrogen bonding with uric acid) have large surface areas that enable fast and effective uric acid scavenging and have a diameter that is compatible with conventional screen-printing and syringe dispensing application techniques.

### Example 1

An aqueous dispersion of polyVDAT nanoparticles was created by synthesizing polyVDAT in a 1 liter glass reactor vessel as follows. 600 grams of deionized water was added to the reactor vessel and heated to 70 degrees Celsius. 20.0 g VDAT (commercially available from TCI America) and 0.2 g 2,2′-azobis-(2-amidinopropane)hydrochloride were dissolved in 250 g dimethyl sulfoxide (DMSO) in a 500 ml round-bottom glass flask which was equipped with magnetic stirrer bars, nitrogen inlets and outlets. Flowing nitrogen was employed to deoxygenate the reactor vessel and the round-bottom flask, which were under agitation.

The solution in the round-bottom flask was then fed into the reactor at a flow rate of approximately 0.8 ml per minute and the polymerization continued for 15 hours. The resulting product was purified by dialysis in cellulose tubing (Sigmal-Aldrich, product car. No. D9777) against DDI water over 5 days with daily change of the water. FIG. 3 indicates that the synthesized irregular-shaped polyVDAT nanoparticles had two populations: one population with a diameter of around 100 nm and the other around 250-300 nm.

### Example 2

The polyVDAT nanoparticle synthesis of example 2 was identical to that of example 1 with the exception that 10 g of VDAT (commercially available from TCI America) and 0.5 g potassium persulfate was dissolved in 150 ml MDSO and fed continuously to the reactor at a flow rate of approximately 0.3 mL per minute. FIG. 4 indicates that the essentially spherical polyVDAT nanoparticles have a diameter of approximately 400 nm.

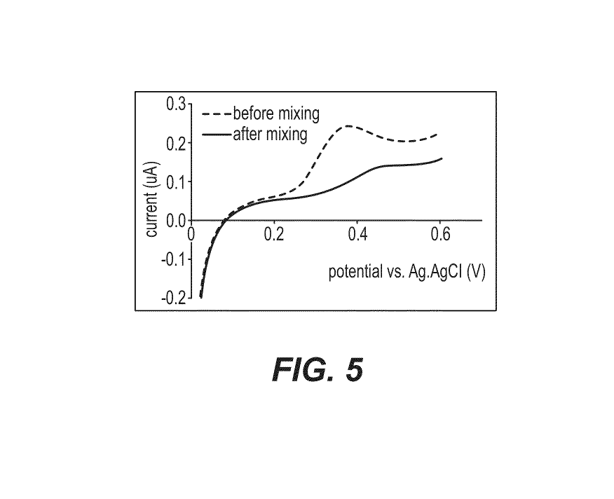

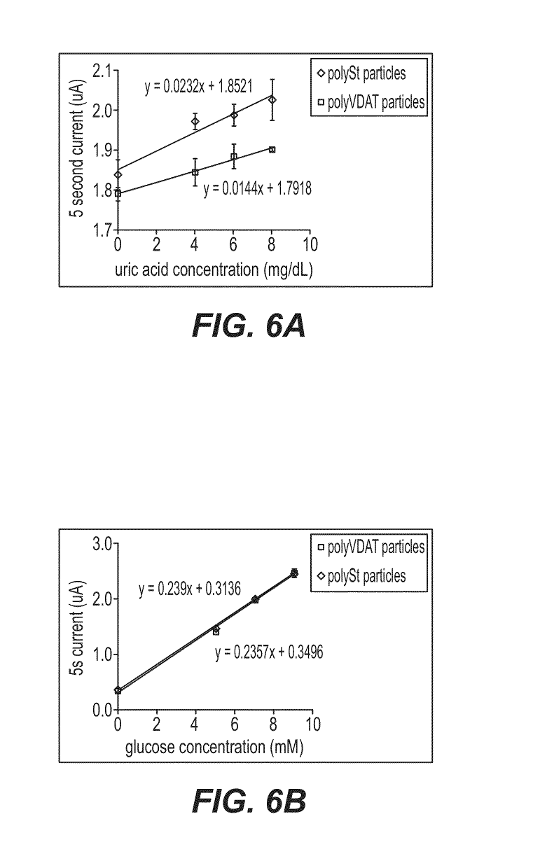

Referring to FIG. 5 through 6B, the synthesized polyVDAT nanoparticles exhibited significant uric acid scavenging characteristics. For example, the cyclic voltammogram of FIG. 5 indicate a significant reduction in the uric acid oxidation peak following the treatment (i.e., mixing) of a uric acid solution with polyVDAT nanoparticles (synthesized per Example 1) before such solution was added to phosphate buffered saline (PBS). It was calculated that the polyVDAT nanoparticles adsorbed approximately 1.0 milligram of uric acid per gram of polyVDAT nanoparticles.

A solution of 2% w/w of dispersion of polyVDAT nanoparticles (created per the synthesis of Example 1) and 0.5% w/w of Pluronic P103 (added as a binder to maintain the integrity of the deposited uric acid scavenger layer) in water was incorporated into an electrochemical-based analytical test strip configured for the determination of glucose in a whole blood sample. The polyVDAT nanoparticles were included in the electrochemical-based analytical test strip as a uric acid scavenger layer with a thickness in the range of 0.5 to 1.5 microns disposed on top of an enzymatic reagent layer (see FIG. 9 described below). Electrochemical test strips with polystyrene particles (diameter of approximately 330 nm) substituted for the polyVDAT nanoparticles were also created as control strips.

The data of FIGS. 6A and 6B indicate that the electrochemical-based analytical test strips according to the present invention (i.e., the test strips with a uric acid scavenger layer containing the polyVDAT nanoparticles) produced electrochemical responses that were significantly less sensitive to uric acid across the tested concentration range than the control strips (about 38% reduction in the slope). However, the experimental slope data for the glucose tests (see FIG. 6B, which were conducted in the absence of uric acid) shows that the strips according to the present invention produced almost the same electrochemical responses as the control strips. These data clearly indicate that the difference in sensitivity to uric acid between the test strips according to the present invention and the control strips mainly results from uric acid adsorption (scavenging) by the polyVDAT nanoparticles rather than any other differences (e.g., diffusion characteristics of the layers, electrode surface areas, etc.) between the two types of strips. Therefore, the presence of polyVDAT nanoparticles in the strips have significantly reduced uric interference and hence, provides for improved analyte determination accuracy.

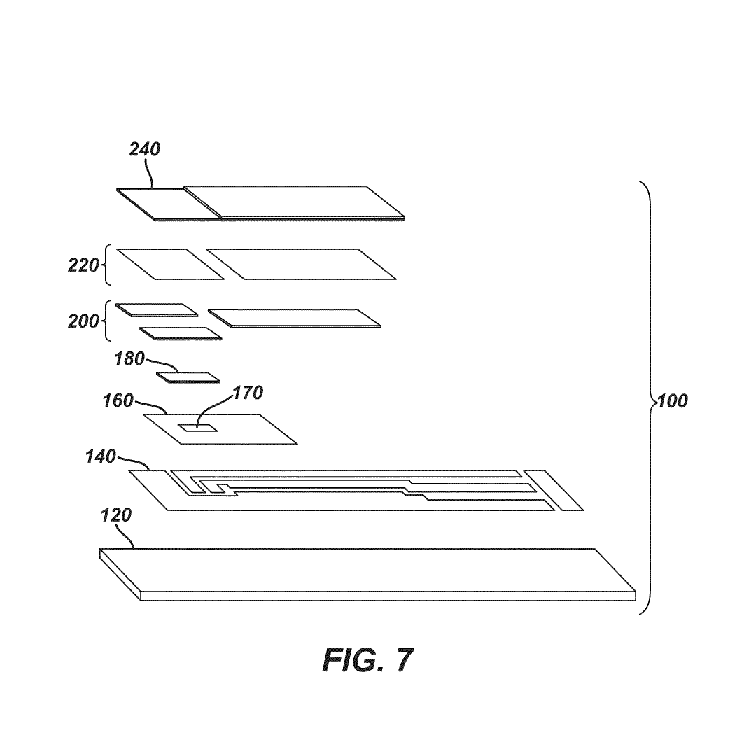

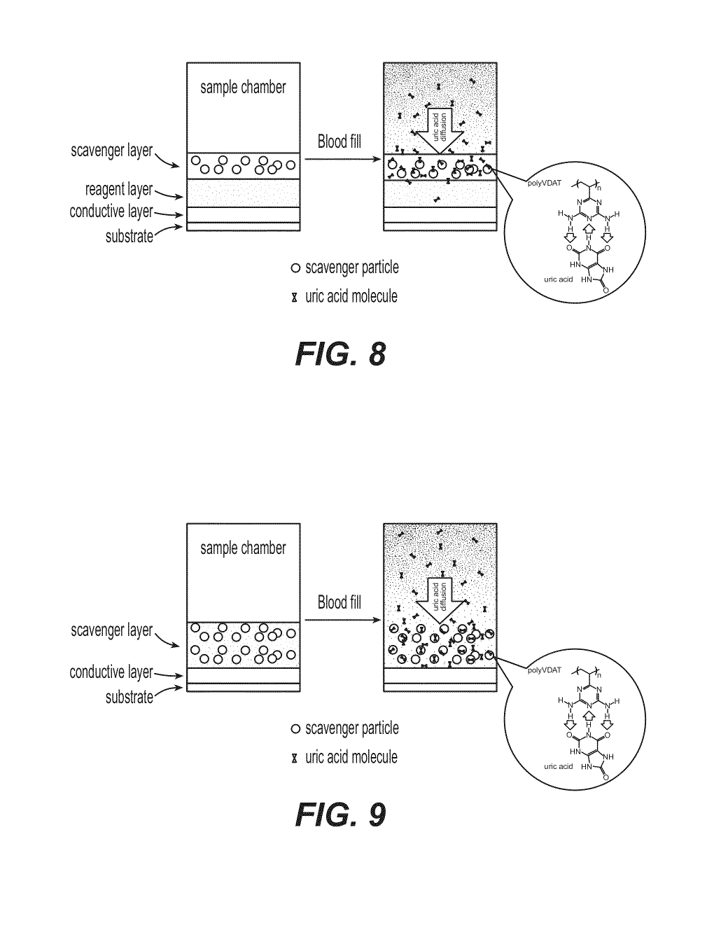

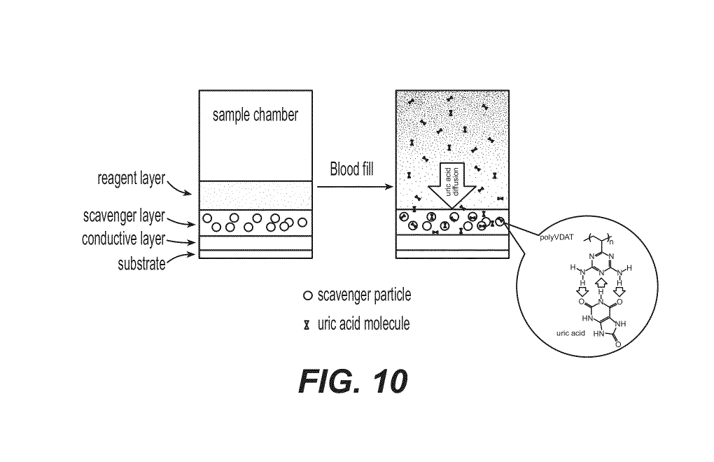

FIG. 7 is a simplified exploded perspective view of an electrochemical-based analytical test strip 100 containing a uric acid scavenger polyVDAT nanoparticle layer according to an embodiment of the present invention. FIG. 8 is a sequence of simplified depictions of an analytical test strip that includes a uric acid scavenger polyVDAT nanoparticle layer disposed above an enzymatic reagent layer in use with a blood sample according to an embodiment of the present invention in use. FIG. 9 is a sequence of simplified depictions of electrochemical-based analytical test strip 100, that includes a combined uric acid scavenger polyVDAT nanoparticle layer and enzymatic reagent layer in use with a blood sample according to an embodiment of the present invention in use. FIG. 10 is a sequence of simplified depictions of an electrochemical-based analytical test strip that includes a uric acid scavenger polyVDAT nanoparticle layer disposed under an enzymatic reagent layer in use with a blood sample according to an embodiment of the present invention in use. In FIGS. 8, 9 and 10, the term “scavenger layer” refers to a uric acid scavenger layer containing polyVDAT nanoparticles, the term “conductive layer” refers to an electrode (e.g., a working electrode), and the term “scavenger particle” refers to a polyVDAT nanoparticle as described herein.

Referring to FIG. 7 through 10, electrochemical-based analytical test strip 100 includes an electrically-insulating substrate layer 120, a patterned conductor layer 140, an insulation layer 160 (with electrode exposure window 170 extending therethrough), a combined enzymatic reagent and uric acid scavenger layer 180, a patterned spacer layer 200, a hydrophilic layer 220 and a top film 240. Patterned conductor layer 140 includes three electrodes portions.

Electrically-insulating substrate layer 120 can be any suitable electrically-insulating substrate known to one skilled in the art including, for example, a nylon substrate, polycarbonate substrate, a polyimide substrate, a polyvinyl chloride substrate, a polyethylene substrate, a polypropylene substrate, a glycolated polyester (PETG) substrate, or a polyester substrate. The electrically-insulating substrate can have any suitable dimensions including, for example, a width dimension of about 5 mm, a length dimension of about 27 mm and a thickness dimension of about 0.5 mm.

Insulation layer 160 can be formed, for example, from a screen printable insulating ink. Such a screen printable insulating ink is commercially available from Ercon of Wareham, Mass. U.S.A. under the name “Insulayer.” Patterned spacer layer 200 can be formed, for example, from a screen-printable pressure sensitive adhesive commercially available from Apollo Adhesives, Tamworth, Staffordshire, UK.

Hydrophilic layer 220 can be, for example, a clear film with hydrophilic properties that promote wetting and filling of electrochemical-based analytical test strip 100 by a fluid sample (e.g., a whole blood sample). Such clear films are commercially available from, for example, 3M of Minneapolis, Minn. U.S.A. Top film 240 can be, for example, a clear film overprinted by black decorative ink. A suitable clear film is commercially available from Tape Specialities, Tring, Hertfordshire, UK.

Combined enzymatic reagent and uric acid scavenger layer 180 can include, in addition to polyVDAT nanoparticles, any suitable enzymatic reagents, with the selection of enzymatic reagents being dependent on the analyte to be determined. For example, if glucose is to be determined in a blood sample, combined enzymatic reagent and uric acid scavenger layer 180 can include glucose oxidase or glucose dehydrogenase along with other components necessary for functional operation. Further details regarding enzymatic reagent layers, and electrochemical-based analytical test strips in general, are in U.S. Pat. No. 6,241,862, the contents of which are hereby fully incorporated by reference.

Combined enzymatic reagent and uric acid scavenger layer 180 contains polyVDAT nanoparticles as illustrated in FIG. 9. Alternatively, a separate uric acid scavenger layer with polyVDAT nanoparticles can be disposed above an enzymatic reagent layer (as depicted in FIG. 8) or between an enzymatic reagent layer and a conductive electrode layer (as depicted in FIG. 10).

The configuration of FIG. 8 wherein a uric acid scavenger layer with polyVDAT nanoparticles is disposed above an enzymatic reagent layer can be particularly beneficial when the enzymatic reagent layer includes a component that oxidizes uric acid. In such a configuration, uric acid is scavenged from a bodily fluid sample prior to a bodily fluid sample reaching the enzymatic reagent layer, thus reducing the interfering effect of the uric acid. The configuration of FIG. 9 can be particularly beneficial with respect to simplicity of biosensor manufacturing since the combined uric acid scavenger layer with polyVDAT nanoparticle and enzymatic reagent layer can be applied to the conductive layer (i.e., electrode) in a single application.

Electrochemical-based analytical test strip 100 can be manufactured, for example, by the sequential aligned formation of patterned conductor layer 140, insulation layer 160 (with electrode exposure window 170 extending therethrough), combined enzymatic reagent and uric acid scavenger layer 180, patterned spacer layer 200, hydrophilic layer 220 and top film 240 onto electrically-insulating substrate layer 120. Any suitable techniques known to one skilled in the art can be used to accomplish such sequential aligned formation, including, for example, screen printing, photolithography, photogravure, chemical vapour deposition and tape lamination techniques.

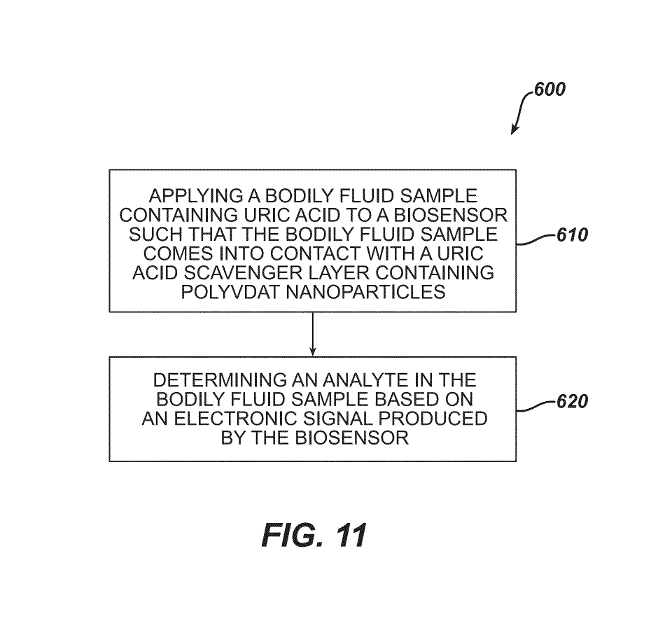

FIG. 11 is a flow diagram depicting stages in a method 600 for determining an analyte (such as glucose) in a bodily fluid sample (e.g., a whole blood sample) containing uric acid according to an embodiment of the present invention. At step 610, method 600 includes applying a bodily fluid sample containing uric acid to a biosensor such that the bodily fluid sample comes into contact with a uric acid scavenger layer containing polymeric vinyl-4,6-diamino-1,3,5-triazine (polyVDAT) nanoparticles. Method 600 further includes determining the analyte in the bodily fluid sample based on an electronic signal produced by the biosensor (see step 620 of FIG. 11).

Once apprised of the present disclosure, one skilled in the art will recognize that method 600 can be readily modified to incorporate any of the techniques, benefits and characteristics of biosensors and aqueous vinyl-4,6-diamino-1,3,5-triazine (VDAT) compositions according to embodiments of the present invention and described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that compositions, devices and methods within the scope of these claims and their equivalents be covered thereby.

What is claimed is:

1. A biosensor comprising:
a substrate;
at least one electrode disposed on the substrate; and
a uric acid scavenger layer containing polymeric nanoparticles including polymerized vinyl-4,6-diamino-1,3,5-triazine (polyVDAT nanoparticles) disposed above the at least one electrode.

2. The biosensor of claim 1 wherein the polyVDAT nanoparticles have a diameter in the range of 30 nanometers to 1000 nanometers.

3. The biosensor of claim 1 wherein the polyVDAT nanoparticles are essentially spherical in shape.

4. The biosensor of claim 1 wherein the polyVDAT nanoparticles include only polymerizedvinyl-4,6-diamino-1,3,5-triazine.

5. The biosensor of claim 4 wherein the polyVDAT nanoparticles are of the following chemical structure: 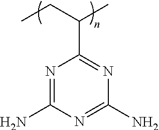
where n is in the range of 15 to 5000.

6. The biosensor strip of claim 1 wherein the polyVDAT nanoparticles are a copolymer of vinyl-4,6-diamino-1,3,5-triazine and one or more monomers.

7. The biosensor of claim 6 wherein the monomer is at least one of styrene and methyl methacrylate.

8. The biosensor of claim 1 wherein the polyVDAT nanoparticles are synthesized from a mixture of vinyl-4,6-diamino-1,3,5-triazine and a crosslinking compound.

9. The biosensor of claim 1 wherein the at least one electrode includes a working electrode and a counter/reference electrode and the uric acid scavenger layer is disposed above at least the working electrode.

10. The biosensor of claim 9 wherein the biosensor is configured as an electrochemical-based analytical test strip and includes an enzymatic reagent layer.

11. The biosensor of claim 10 wherein the uric acid scavenger layer containing polyVDAT nanoparticles is disposed above the enzymatic reagent layer and the working electrode.

12. The biosensor of claim 10 wherein the uric acid scavenger layer containing polyVDAT nanoparticles is disposed between the enzymatic reagent layer and the working electrode.

13. The biosensor of claim 10 wherein the enzymatic reagent layer that is integrated with the scavenger layer containing polyVDAT nanoparticles.

14. The biosensor of claim 10 wherein the electrochemical-based analytical test strip is configured for the determination of glucose in a whole blood sample.

15. The biosensor of claim 1 wherein the polyVDAT nanoparticles are irregular in shape.

16. An aqueous vinyl-4,6-diamino-1,3,5-triazine (VDAT) composition for use in analytical test strips, the aqueous vinyl-4,6-diamino-1,3,5-triazine (VDAT) composition comprising:
polymeric nanoparticles including polymerized vinyl-4,6-diamino-1,3,5-triazine (polyVDAT nanoparticles); and
water,
wherein the polyVDAT nanoparticles are present as a dispersion in the water.

17. The aqueous vinyl-4,6-diamino-1,3,5-triazine (VDAT) composition of claim 16 wherein the polyVDAT nanoparticles have a diameter of less than 1000 nanometers.

18. The aqueous vinyl-4,6-diamino-1,3,5-triazine (VDAT) composition of claim 16 wherein the polyVDAT nanoparticles have a diameter in the range of 30 nanometers to 1000 nanometers.

19. The aqueous vinyl-4,6-diamino-1,3,5-triazine (VDAT) composition of claim 16 wherein the polyVDAT nanoparticles are essentially spherical in shape.

20. The aqueous vinyl-4,6-diamino-1,3,5-triazine (VDAT) composition of claim 16 wherein polyVDAT nanoparticles are irregular in shape.

21. The aqueous vinyl-4,6-diamino-1,3,5-triazine (VDAT) composition of claim 16 further including a binder.

22. The aqueous vinyl-4,6-diamino-1,3,5-triazine (VDAT) composition of claim 16 wherein the aqueous vinyl-4,6-diamino-1,3,5-triazine (VDAT) composition has a pH between 4.0 and 14.0.

23. The aqueous vinyl-4,6-diamino-1,3,5-triazine (VDAT) composition of claim 16 wherein the polyVDAT nanoparticles include only polymerized vinyl-4,6-diamino-1,3,5-triazine.

24. The aqueous vinyl-4,6-diamino-1,3,5-triazine (VDAT) composition of claim 16 wherein the polyVDAT are a copolymer of vinyl-4,6-diamino-1,3,5-triazine and one or more monomers.

25. The aqueous vinyl-4,6-diamino-1,3,5-triazine (VDAT) composition of claim 16 wherein the polyVDAT nanoparticles are synthesized from a mixture of vinyl-4,6-diamino-1,3,5-triazine and a crosslinking compound.

26. A method for determining an analyte in a bodily fluid sample containing uric acid, the method comprising:
applying a bodily fluid sample containing uric acid to a biosensor such that the bodily fluid sample comes into contact with a uric acid scavenger layer containing polymeric nanoparticles that include polymerized vinyl-4,6-diamino-1,3,5-triazine (polyVDAT nanoparticles); and
determining the analyte based on an electronic signal produced by the biosensor.

27. The method of claim 26 wherein the polyVDAT nanoparticles have a diameter of less than 1000 nanometers.

28. The method of claim 26 wherein the polyVDAT nanoparticles have a diameter in the range of 30 nanometers to 1000 nanometers.

29. The method of claim 26 wherein the polyVDAT nanoparticles are essentially spherical in shape.

30. The method of claim 26 wherein the biosensor is configured as an electrochemical-based analytical test strip.

31. The method of claim 30 wherein the bodily fluid sample is a whole blood sample and the analyte is glucose.

32. The method of claim 26 wherein the polyVDAT nanoparticles include only polymerized vinyl-4,6-diamino-1,3,5-triazine.

33. The method of claim 26 wherein the polyVDAT nanoparticles are a copolymer of vinyl-4,6-diamino-1,3,5-triazine and one or more monomers.

34. The method of claim 26 wherein the polyVDAT nanoparticles are synthesized from a mixture of vinyl-4,6-diamino-1,3,5-triazine and a crosslinking compound.

35. The method of claim 26 wherein the polyVDAT nanoparticles are of the following chemical structure: 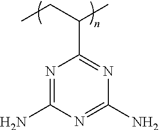
where n is in the range of 15 to 5000.

36. The method of claim 26 wherein the polyVDAT nanoparticles are irregular in shape.
